(19) United States
(12) Patent Application Publication
(10) Pub. No.: US 2010/0076093 A1
(43) Pub. Date: Mar. 25, 2010

(54) USE OF ERGOTHIONEINE AS A PRESERVATIVE IN FOODS AND BEVERAGES

(76) Inventors: Robert B. Beelman, University Park, PA (US); Marvin S. Hausman, Sherwood, OR (US)

Correspondence Address: MCKEE, VOORHEES & SEASE, P.L.C., 801 GRAND AVENUE, SUITE 3200, DES MOINES, IA, 50309-2721, US

(21) Appl. No.: 12/529,859

(22) PCT Filed: Mar. 7, 2008
(86) PCT No.: PCT/US08/56234

§ 371 Date: Dec. 1, 2009

Related U.S. Application Data
(60) Provisional application No. 60/893,511, filed on Mar. 7, 2007.

(51) Int. Cl. A61K 47/22; C12H 1/14; A23L 2/44; A23L 3/3535; C11B 5/00; A23K 1/16; C12G 1/12

(52) U.S. Cl. 514/784; 426/271; 426/547

(57) ABSTRACT

The invention relates to the novel use of ergothioneine and preferably, L ergothioneine, as a nutritional additive and preservative in foods, medicines, and/or beverages. According to the invention, the powerful antioxidant ergothioneine was found to be very stable over time in and to have no deleterious effects on taste or consistency of food and beverages even when stored, over a period of several years. In a preferred embodiment, ergothioneine may be used as a replacement for all or part of the antimicrobial/preservative sulfur dioxide or other sulfites traditionally used in the wine making process.

### BACKGROUND OF THE INVENTION

Chemical preservatives such as sulfur dioxide help keep food fresh. Preservatives can be categorized into three general types: antimicrobials that inhibit growth of bacteria, yeasts, or molds; antioxidants that slow air oxidation of fats and lipids, which leads to rancidity; and a third type that blocks the natural ripening and enzymatic processes that continue to occur in foodstuffs after harvest.

Sulfur dioxide is a commonly used preservative as it serves all three functions, and its related compounds, sulfites are found in foods, alcoholic drinks (especially wines), and even in medications.

Sulfites inhibit microbial growth through a number of actions, they react with the energy currency of the cell, adenosine triphosphate; inhibit some metabolic pathways; and block cellular transport systems. Other antimicrobials alter microbial membrane or cell wall permeability or destroy the genetic material.

In addition to its antimicrobial action, sulfur dioxide inhibits degradation reactions in fruits, by blocking both enzymatic browning and a nonenzymatic browning reaction between reducing sugars and amino acids.

About 1% to 2% of people will have an allergic reaction to sulfites, which can consist of nasal congestion and sneezing, skin hives, or wheezing and difficulty breathing. People who have asthma and/or allergies to aspirin are particularly sensitive to sulfites and could even have a serious anaphylactic reaction, in which there is severe swelling of the throat, tongue, and airway, which obstructs breathing and can lead to death.

Examples of antimicrobials include propionic acid, which occurs naturally in strawberries, apples, violet leaves, grains, and cheese. This acid is effective against bread molds and the spores of the bacterium Bacillus mesentericus, which cause an inedible condition in baked goods called rope.

Other weak organic acid antimicrobials include benzoates, found naturally in cranberries, and sorbates. Because these compounds work best at a low pH—in the range that excludes much bacterial growth—they are used primarily as antifungals. Esters of p-hydroxybenzoic acid, also known as parabens, are similar to benzoic acid but effective at a higher pH. Many beverages, jams, pickled products, salads, cheeses, meats, and margarines contain benzoates or sorbates.

Nitrites and nitrates are the food industry's primary chemical defense against the bacterium Clostridium botulinum. They also impart a pink, fresh hue to cured meat. Nitrates readily convert to nitrites, which then react with the protein myoglobin to form nitric oxide myoglobin. During cooking, this is converted to nitrosohemochrome, a stable, pink pigment. In the absence of nitrates or nitrites, meat turns brown. However, nitrites react with amino acids to form the cancer causing agents, nitrosamines.

A third group of preservatives targets enzymes in the food itself that continue to metabolize after harvest. The enzyme polyphenoloxidase, for example, goes to work as soon as an apple or potato is cut. It browns the exposed surface. Acids such as citric acid and ascorbic acid (vitamin C) and erythorbic acid inhibit phenolase by making the pH uncomfortably low for the enzyme.

And metal-chelating agents such as EDTA (ethylenediamine tetraacetic acid) can remove the metal cofactors that many enzymes need. Chelators also make it difficult for plant bacterial and fungal enzymes to carry on.

As can be seen there is a need in the art for other food preservatives, preferably those that are in natural products. Some of the newest antimicrobials have been found in microorganisms themselves as they form their own chemical defenses when competing with each other for space and nutrients. For example, nisin and natamycin—cheese preservatives called bacteriocins—are harvested from microorganisms. Other potential natural preservative sources include honey, milk, and even dried plums as scientists seek new sources and combinations of safe, effective preservatives.

It is an object of the present invention to provide a safe effective naturally occurring preservative that is stable, and does not adversely affect the taste of food products.

### SUMMARY OF THE INVENTION

According to the invention, Applicant's have found that the naturally occurring compound, ergothioneine has the ability to act as an antioxidant and chelator of heavy metals and may be used as a for food preservative for food, beverages, medicines and the like.

The invention thus relates to the novel use of ergothioneine and preferably, L ergothioneine, (also known as thiotane or thiotaine) having a chemical formula as seen in FIG. 1, as a preservative in foods, medicines, and/or beverages. Ergothioneine was found to be very stable over time in and to have no deleterious effects on taste and consistency of food and beverages even when stored over a period of several years.

The invention includes a method of preserving beverages, medicines and other food stuffs by adding to the same an effective amount of ergothioneine, preferably L-ergothioneine. The invention also includes foods, beverages and medicines so modified, as well as methods of maintaining and improving overall health by administration of products fortified with this nutritionally valuable antioxidant.

In one embodiment, the ergothioneine is a replacement for all or part of sulfur dioxide or other sulfites used as a food preservative and can be used as an anti-microbial, and antioxidant without the allergic effects of sulfur and sulphites.

According to the invention, ergothioneine, preferably L-ergothioneine, may be used as a replacement for all or part of other known preservatives such as ascorbic acid, sodium nitrates, propionic acid, sorbic acid, benzoic acid, sodium erythorbate, erythorbic acid, ascorbic acid, sodium succinate, grape seed extract, pine bark extract, apple extract tea proplyphenols, succinic acid and preservatives like parabens, and sodium dehydro acetate.

Not only is ergothioneine useful as a food preservative, is a powerful antioxidant with health benefits for the consumer of foodstuffs and beverages so preserved. Thus, the composition, in addition to helping preserve the food or beverage product, also makes the product more nutritionally healthy. Thus another aspect of the invention is the use of ergothioneine, particularly, L-ergothioneine as an additive to food and beverages to add to the health benefits of the same.

Numerous studies have shown that consuming fruits and vegetables which are high in antioxidants may reduce the risk of developing chronic diseases. Ergothioneine, highly protective, nontoxic, naturally occurring compound with strong antioxidant properties and which provides cellular protection within the human body. One unit of L-Ergothioneine is approximately equivalent to 7000 units of Vitamin E. It is readily water soluble, reaches near millimolar concentrations in selected tissues, and stimulates the natural antioxidant defenses within cells.

The benefits of natural antioxidants such as vitamin C and vitamin E in cancer, aging and general health are well known. Newer natural antioxidants such as pyrogallol, lipoic acid and ubiquinone are now being introduced into the market. L-ergothioneine is unique among antioxidants in that it chelates heavy metal, while protecting cells (principally erythrocytes) from damage and has its own transport system for uptake into cells, See PCT published application number PCT WO 2005/116657, the disclosure of which is hereby incorporated in its entirety by reference.

While not wishing to be bound by any theory, it is postulated that ergothioneine which naturally occurs in red wine, may be responsible for some of the healthy effects of red wine noted of late, which has been attributed to the flavonoids and antioxidants present in red wine. They include the effects of helping to reduce the production of LDL, low density lipoprotein—sometimes referred to as “bad” cholesterol. Red wine has also been shown to have the effect of increasing HDL, high density lipoprotein, the so-called “good” cholesterol. These combined effects help to prevent blood clots and improve the lipid profile overall.

### DESCRIPTION OF THE FIGURES

FIG. 1 is a depiction of the chemical structure of ergothioneine.

### DETAILED DESCRIPTION OF THE INVENTION

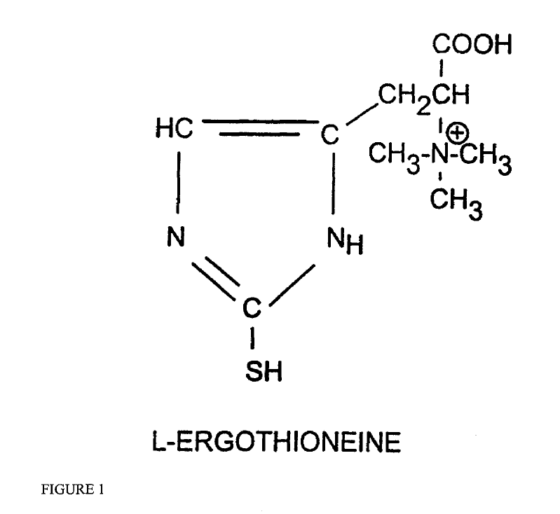

Chemically L-ergothioneine (C₉H₁₅N₃O₂S.H₂O) corresponds to the betaine of 2-thio-L-histidine, it is the only known naturally occurring 2-thio-imidazole amino acid to date. Its formula is shown in FIG. 1. The compound is also known as thiotane or thiotaine, as used herein, the term L-ergothioneine shall also include thiotaine or thiotaine as exemplified by the compound (C₉H₁₅N₃O₂S.H₂O) for the methods and compositions of the invention. The compound is extremely hydrophilic with a solubility limit of 0.9M at room temperature.

Ergothioneine is a phytonutrient and naturally occurring antioxidant that is very stable in the body. No toxicity to this compound has been shown. It is synthesized in fungi and a few bacteria, and present in both plants and animals. Animals are unable to synthesize L-ergothioneine and must obtain it from dietary sources. It is readily absorbed and is active in most mammalian tissues, concentrating especially in the liver, where it prevents certain types of free-radical-induced damage to cell membranes and organelles. For example, exogenous L-ergothioneine has been shown to prevent lipid peroxidation by toxic compounds in the liver tissue of rats. In a recent study comparing the inhibition of lip peroxide (“LPO”) formation by various compounds in mouse liver, L-ergothioneine both inhibited LPO formation and enhanced the decomposition of existing LPO.

Additionally, L-ergothioneine serves as an antioxidant and a cellular protector against oxidative damage. The antioxidant properties of L-ergothioneine include: a scavenger of strong oxidants; chelation of various divalent metallic cations; and plays a key role in the oxidation of various hemoproteins. L-ergothioneine has been shown to inhibit the damaging effects caused by the oxidation of iron-containing compounds, such as hemoglobin and myoglobin. These molecules are important in the body as carriers of oxygen, but because they contain divalent iron, they can interact with hydrogen peroxide via the Fenton reaction to produce the even more damaging hydroxyl radical. This has been suggested as a mechanism by which damage occurs during so-called reperfusion injury.

Studies have shown that ergothioneine is a powerful scavenger of hydroxyl radicals, but unlike other scavengers, ergothioneine is able to inhibit iron and copper-ion dependent generation of hydroxyl radicals (Alanmu, et al 1991). Also, ergothioneine has the ability to complex with divalent metal ions; such as copper, cadmium and mercury (Motohashi et al 1976). Ergothioneine is an excellent chelator of divalent metal ions. This is particularly important as iron and copper ions play a significant role in catalytic browning and instability issues in wine and other foods.

Processes for obtaining ergothioneine and L-ergothioneine synthetically and as purified from natural sources such as pig blood, or grains are known in the art and embodied, for example in U.S. Pat. No. 5,438,151, incorporated herein by reference. The composition is also commercially available from Oxis, International, Foster City, Calif., and from Toronto Research Chemicals, Inc., North York, Ontario, Canada. It is also commonly purified from filamentous fungi such as mushrooms, particularly mycelia. It may also be purified from liquid culture of mycelia of filamentous fungi from the mycelia, or as excreted into the culture supernatant.

According to the invention, traditional food additives, such as sulfur dioxide, ascorbic acid or erythorbic acids and/or their salts may be replaced partially or completely by ergothioneine, preferably L-ergothioneine. The addition of ergothioneine to foodstuffs and beverages not only can have an antioxidant effect but also an antimicrobial effect as well. Ergothioneine also has a phytonutrient benefits that may make the food more nutritional.

For example, in the wine making process sulfur dioxide is added to reduce oxidation and preserve wine during storage and fermentation. It is added at very high levels, 180 ppm to prevent rot and hoer deterioration. By the time the wine reaches the consumer, the sulfur dioxide has degraded and is present only at 60-60 ppm. The CU and most countries have established legal limits of sulfur dioxide limits in wine. In the stomach, much of the bound sulfur dioxide will be released by the acidity and warmth of the digestive system, becoming toxic to human health if above a safe limit. Typical limits are 260 mg/lit for red wines and 210 mg/lit for white wines. Sulfur dioxide is added at many stages during the wine making process and serves two, basic purposes, Firstly, it is an anti-microbial agent, and as such is used to help curtail the growth of undesirable fault producing yeasts and bacteria. Secondly, it acts as an antioxidant, safeguarding the wine's fruit integrity and protecting it against browning. According to the invention, all or part of the sulfur dioxide added to wine may be replaced with L-ergothioneine. For example wine makers concerned with overall texture of wine may want to hang and store grapes before crushing, ergothioneine could be added at this stage to help with the storage and to prevent breakdown of the grapes. L-ergothioneine may be added and used at different temperatures to improve overall effectiveness as it is more stable than sulfur dioxide. Ergothioneine could also be used to stabilize the lees, the sediment containing the grape skins from wine production.

As used herein the term ergothioneine shall be interpreted to include variants, homologs, optical isomers and the like which retain the antioxidant activity of ergothioneine or L-ergothioneine as demonstrated and described herein.

Ergothioneine from any source may be used according to the invention.

The preservative compositions of the invention comprise ergothioneine alone or in combination with other excipients, carriers, fillers additives and the like. In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropyl cellulose; or combinations thereof such methods. In many cases, it will be preferable to include isotonic agents, such as, for example, sugars, sodium chloride or combinations thereof.

In certain embodiments the preservative composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. Typical formulae for compositions are, well known in the art. In addition to proteinaceous and farinaceous materials, the compositions of the invention generally may include vitamins, minerals, and other additives such as flavorings, preservatives, emulsifiers and humectants. The nutritional balance, including the relative proportions of vitamins, minerals, protein, fat and carbohydrate, is determined according to dietary standards known in the veterinary and nutritional art.

The preservative composition of the present invention can further comprise a wide range of other optional ingredients. Non limiting examples of additional components include animal protein, plant protein, farinaceous matter, vegetables, fruit, egg-based materials, undenatured proteins, food grade polymeric adhesives, gels, polyols, starches, gums, flavorants, seasonings, salts, colorants, time-release compounds, minerals, vitamins, antioxidants, prebiotics, probiotics, aroma modifiers, textured wheat protein, textured soy protein, textured lupin protein, textured vegetable protein, breading, comminuted meat, flour, comminuted pasta, water, and combinations thereof.

Also useful herein, as an optional ingredient, is a filler. The filler can be a solid, a liquid or packed air. The filler can be reversible (for example thermo-reversible including gelatin) and/or irreversible (for example thermo-irreversible including egg white). Non limiting examples of the filler include gravy, gel, jelly, aspic, sauce, water, air (for example including nitrogen, carbon dioxide, and atmospheric air), broth, and combinations thereof.

Any food, beverage or medicine in need of preservatives, particularly antioxidants to enhance stability, shelf life and the like may be treated with the methods and compositions of the invention. Some non-limiting examples include canned, frozen, dried, or fresh fruits and vegetables or products containing the same, wines (red or white), pet foods, fruit juices, food colorings and dyes, vegetable oils, butter, meats, cereals, chewing gum, baked goods, snack foods, dehydrated potatoes, beer animal feed, food packaging, cosmetics, rubber products, and petroleum products, cookies, crackers, beet sugar, and pie dough.

Preservative compositions according to the present invention may by applied to a product in a number of ways. For example, such compositions may be sprayed, injected, dipped or poured directly onto products. Alternatively, preservative compositions may be frozen and products may be placed in contact with the frozen preservative compositions. Further, preservative compositions may be spray dried, freeze-dried and/or powdered and then applied to products. Preservative compositions may be added to a finished product or may be added at any step in the production processes of a product.

Alternatively, the preservative of the invention as described above can be individually or collectively added to the final product or to what becomes the final product, or in a process of making the final product, either separately or all together at once.

The following examples are non-limiting and are only for purposes of illustration. All references cited herein are hereby incorporated in their entirety by reference.

### EXAMPLES

According to the invention, sulfur dioxide was replaced in whole or in part with L-ergothioneine in Pinot noir wines at bottling. After 18 months of storage, it was found that the ergothioneine was still present in the wine and did not result in any deleterious impact on taste of the resultant wine.

TABLE 1
Addition of various concentration of SO₂ and L-ergothioneine (ERGO)
to Pinot noir wines at bottling and concentration of ERGO and color
values found in the wines after 18 months of bottle storage.

| SO₂ Added/ERGO | ERGO Found | Color | | | Abs |
|---|---|---|---|---|---|
| Added (mg/L) | (mg/L) | L | a | b | (520/420) |
| 0/0 | 7.2 | 24.09 | 0.74 | 0.04 | 1.04 |
| 30/0 | 7.3 | 24.07 | 0.68 | 0.02 | 1.08 |
| 70/0 | 7.5 | 24.09 | 0.72 | 0.04 | 1.09 |
| 0/10 | 8.8 | 24.11 | 0.75 | 0.09 | 1.06 |
| 0/30 | 29.8 | 24.07 | 0.76 | 0.03 | 1.04 |
| 0/50 | 55.4 | 24.1 | 0.82 | 0.09 | 1.07 |
| 0/70 | 81.2 | 24.08 | 0.74 | 0.03 | 1.04 |
| 10/60 | 77 | 24.12 | 0.77 | 0.08 | 1.14 |
| 20/50 | 72.4 | 24.12 | 0.79 | 0.07 | 1.14 |
| 30/40 | 55.5 | 24.08 | 0.65 | 0.02 | 1.11 |
| 40/30 | 44.9 | 24.11 | 0.79 | 0.05 | 1.13 |
| 50/20 | 37.8 | 24.14 | 0.85 | 0.08 | 1.11 |

What is claimed is:

1. A method of preserving food, beverages or medicines comprising:
adding to said food, beverage, or medicine an effective amount of ergothioneine.

2. The method of claim 1 wherein said ergothioneine is L-ergothioneine.

3. The method of claim 1 wherein said ergothioneine replaces all or part of an antioxidant composition.

4. The method of claim 1 wherein said ergothioneine replaces all or part of an anti-microbial composition.

5. The method of claim 1 wherein said ergothioneine is synthetically derived.

6. The method of claim 1 wherein said ergothioneine is purified from a natural source.

7. The method of claim 6 wherein said natural source is filamentous fungi.

7. The method of claim 6 wherein said natural source is mushrooms.

7. The method of claim 6 wherein said natural source is the mycelium of filamentous fungi.

8. The method of claim 6 wherein said natural source is blood.

9. The method of claim 1 wherein said ergothioneine replaces part of sulfur dioxide.

10. The method of claim 1 wherein said ergothioneine is recombinant.

11. The method of claim 1 wherein said food, beverage or medicine is selected from the group consisting of: include canned, frozen, dried, or fresh fruits and vegetables or products containing the same, wines (red or white), pet foods, fruit juices, food colorings and dyes, vegetable oils, butter, meats, cereals, chewing gum, baked goods, snack foods, dehydrated potatoes, beer, animal feed, food packaging, cosmetics, rubber products, and petroleum products, cookies, crackers, beet sugar, and pie dough.

12. The method of claim 1 wherein said beverage is wine.

13. The method of claim 1 wherein said food is fruit or vegetables or products containing the same.

14. The method of claim 1 wherein said product is animal feed.

15. A preservative composition for food, beverages, or medicines comprising: L-ergothioneine and a carrier.

16. The preservative composition of claim 15 further comprising sulfur dioxide.

17. A food, beverage or medicine product preserved by the method of claim 1.

18. A method of preserving and nutritionally enhancing foodstuffs such as food, beverages and the like comprising:
adding to said foodstuffs an effective amount of L-ergothioneine.

19. The method of claim 18 wherein said foodstuff is wine.

20. The method of claim 18 wherein said L-ergothioneine acts as an antioxidant.

21. The method of claim 18 wherein said L-ergothioneine acts as an anti-microbial compound.

22. The method of claim 18 wherein said L-ergothioneine is synthetically derived.

23. The method of claim 18 wherein said L-ergothioneine is purified from a natural source.

7. The method of claim 18 wherein said natural source is filamentous fungi.

7. The method of claim 18 wherein said natural source is mushrooms.

7. The method of claim 18 wherein said natural source is the mycelium of filamentous fungi.

25. The method of claim 23 wherein said natural source is blood.

26. The method of claim 18 wherein said ergothioneine replaces part of sulfur dioxide.

27. The method of claim 18 wherein said ergothioneine is recombinant.

28. The method of claim 18 wherein said feed, beverage or medicine is selected from the group consisting of: include canned, frozen, dried, or fresh fruits and vegetables or products containing the same, wines (red or white), pet foods, fruit juices, food colorings and dyes, vegetable oils, butter, meats, cereals, chewing gum, baked goods, snack foods, dehydrated potatoes, beer, animal feed, food packaging, cosmetics, rubber products, and petroleum products, cookies, crackers, beet sugar, and pie dough.

29. The method of claim 28 wherein said food is a fruit or vegetable product or a product containing the same.

30. A method for preserving wine comprising:
adding to said wine in the bottling process a preservative composition comprising L-ergothioneine.

31. The method of claim 30 wherein said preservative composition replaces all or part of a sulfur dioxide preservative composition.

32. A method of enhancing the nutritional benefit of a food or beverage comprising:
combining said food or beverage with the antioxidant ergothioneine wherein the ergothioneine does not have a deleterious effect on taste or storage of said food or beverage.

33. The method of claim 32 wherein said ergothioneine is L-ergothioneine.

34. A food or beverage produced by the method of claim 32.
